(19) United States
(12) Patent Application Publication
(10) Pub. No.: US 2022/0241337 A1
(43) Pub. Date: Aug. 4, 2022

(54) PREPARING TISSUES FOR DELIVERY OF THERAPEUTIC AND DIAGNOSTIC AGENTS AND DELIVERING THE AGENTS

(71) Applicant: The Trustees of Columbia University in the City of NewYork, New York, NY (US)

(72) Inventor: Akiva Mintz, Paramus, NJ (US)

(21) Appl. No.: 17/432,704

(22) PCT Filed: Feb. 21, 2020
(86) PCT No.: PCT/US2020/019191

§ 371 Date: Aug. 20, 2021

Related U.S. Application Data
(60) Provisional application No. 62/809,068, filed on Feb. 22, 2019.

(51) Int. Cl. A61K 35/28; A61K 31/495; A61P 35/00; A61K 48/00

(52) U.S. Cl. CPC A61K 35/28; A61K 48/0025; A61P 35/00; A61K 31/495

(57) ABSTRACT

Methods of preparing tissues for treatment or imaging and for treating or imaging tissues in a subject in which therapeutic or imageable molecules operably associated with a heat-inducible promoter and an HSP-inducing molecule are administered to the subject.

### REFERENCE TO RELATED APPLICATIONS

This application is a National Stage Application of International Application No. PCT/US2020/019191 filed on Feb. 21, 2020, which claims priority from U.S. provisional patent application No. 62/809,068, filed Feb. 22, 2019, which is incorporated herein in its entirety for all purposes as if fully set forth herein.

### GOVERNMENT RIGHTS

This invention was made with government support under CA207206 awarded by the National Institutes of Health. The government has certain rights in the invention.

### TECHNICAL FIELD

The present disclosure concerns methods for delivering active or therapeutic agents such as stem cells or DNA or diagnostic agents such as imageable molecules to a tissue of interest, such as neoplastic or non-neoplastic tissue in the brain or elsewhere in the body

### BACKGROUND

The following discussion is provided merely to aid the reader in understanding the disclosure and is not admitted to describe or constitute prior art.

The selective delivery of therapeutic molecules to tumor cells, either via stem cells, T cells or nucleic acids is a long sought goal of cancer therapy.

United States patent publication 2016/0324989 (application Ser. No. 15/110,211 filed Jan. 13, 2015) and Xiong et al.—“Remote spatiotemporally controlled and biologically selective permeabilization of the blood-brain barrier”—Journal of Controlled Release 2217 (2015) 113-120 describe methods of preparing for treatment and treating tissues in a subject. These publications are incorporated by reference herein in their entirety.

### SUMMARY

In one aspect, the present disclosure provides a method of preparing a tissue for therapeutic treatment or diagnostic treatment (e.g., imaging) in a subject in need thereof, comprising: (a) parenterally administering to the subject either i) a cell-based carrier (such as stem cells or T cells) that migrates to said tissue, said cell-based carrier containing a recombinant nucleic acid or ii) a nucleic acid that migrates to said tissue, said recombinant nucleic acid comprising an encoding nucleic acid encoding a chemokine, cytokine, therapeutic molecule, or imageable molecule operably associated with a heat-inducible promoter; and (b) administering to said subject an amount of an HSP-inducing molecule sufficient to induce the expression of said chemokine, cytokine, therapeutic molecule, or imageable molecule in an amount effective to enhance the permeability of the blood-brain barrier or tumor vasculature, enhance the migration of therapeutic stem cells subsequently administered parenterally to said subject, or elicit a therapeutic effect of said therapeutic molecule or elicit an imaging effect of said imageable molecule.

In another aspect, the present method provides engineered cell-based therapies (for example, using stem cells or T cells) that express cytokines in order to increase the permeability of the blood-brain barrier to allow systemic therapies or imaging agents to access a tumor in the brain.

In still another aspect, the present method provides engineered cell based therapies (for example, using stem cells or T cells) that express cytokines under control of an HSP promoter in order to increase tumor vessel permeability to allow greater access of systemic therapies or imageable molecules to the tumor.

As discussed below, the present invention provides methods of preparing for treatment or diagnostic imaging, and methods of treating or imaging, a tissue in a subject in need thereof. When considered together, the methods comprise the steps of:

(a) parenterally administering to the subject either i) a first cell-based carrier (such as stem cells or T cells) that migrates to said tissue, said cell-based carrier containing a first recombinant nucleic acid or ii) a second recombinant nucleic acid that migrates to said tissue, said first and second recombinant nucleic acids comprising an encoding nucleic acid encoding a chemokine, cytokine, therapeutic molecule, or imageable molecule operably associated with a heat-inducible promoter;
(b) administering to said subject an amount of a first HSP-inducing molecule (for example, a chemotherapeutic) sufficient to induce the expression of said chemokine, cytokine, or therapeutic molecule, therein in an amount effective to enhance the migration of therapeutic stem cells subsequently parenterally administered to said subject, elicit an anti-tumor response, or render the subject's blood brain barrier or tumor vasculature more permeable;
(c) parenterally administering to the subject either i) a second cell-based carrier (such as stem cells or T cells) that migrates to said tissue, said cell-based carrier containing a third recombinant nucleic acid or ii) a fourth recombinant nucleic acid that migrates to said tissue, said third and fourth recombinant nucleic acids comprising a nucleic acid encoding a chemokine, cytokine, or therapeutic molecule operably associated with a heat-inducible promoter; and then optionally (but in some embodiments preferably);
(d) administering to said subject an amount of a second HSP inducing molecule (such as a chemotherapeutic) sufficient to induce the expression of said therapeutic agent therein in a treatment-effective amount.

A further aspect of the invention is a method of increasing blood-brain barrier permeability of selected brain tissue in a subject in need thereof, comprising:

(a) parenterally administering to the subject a cell-based carrier (such as stem cells) that migrates to the brain tissue, said cell-based carrier containing a recombinant nucleic acid, said recombinant nucleic acid comprising a nucleic acid encoding a blood brain barrier-opening protein or peptide operably associated with a heat-inducible promoter; and

(b) administering an HSP-inducing molecule sufficient to induce the expression of said blood brain barrier-opening protein or peptide in an amount effective to increase the permeability of the blood-brain barrier in said selected brain tissue.

In a further aspect the invention provides a method of increasing tumor permeability to systemic therapies in a subject in need thereof, comprising: (a) parenterally administering to the subject either i) a cell-based carrier (such as stem cells or T cells) that migrates to said tissue, said cell-based carrier containing a first recombinant nucleic acid or ii) a second recombinant nucleic acid that migrates to said tissue, said first and second recombinant nucleic acids comprising an encoding nucleic acid encoding a tumor endothelial-opening molecule operably associated with a heat-inducible promoter; and then (b) administering an HSP-inducing molecule sufficient to induce the expression of said tumor endothelial-opening molecule in an amount effective to increase the permeability of the tumor to systemic therapies and diagnostic agents such as imaging agents.

In a further aspect, the disclosure provides a method of treating a tissue in a subject in need thereof, comprising: (a) parenterally administering to a subject a therapeutic cell-based carrier or a nucleic acid that migrates to said tissue, said cell-based carrier or nucleic acid containing a recombinant nucleic acid, said recombinant nucleic acid comprising an encoding nucleic acid encoding a therapeutic agent operably associated with a heat-inducible promoter; and then (b) administering to said subject an amount of an HSP-inducing molecule sufficient to induce the expression of said therapeutic agent therein in a treatment-effective amount.

The disclosures of all patent references and publications cited herein are incorporated herein by reference in their entirety as if fully set forth herein.

### DETAILED DESCRIPTION

The present invention is primarily concerned with the treatment of human subjects, but the invention may also be carried out on animal subjects, particularly mammalian subjects such as dogs, cats, livestock and horses for veterinary purposes. While subjects may be of any suitable age, the subjects are in some embodiments neonatal, infant, juvenile, adolescent, adult, or geriatric subjects.

“Treat” as used herein refers to any type of treatment that imparts a benefit to a patient, particularly delaying or retarding the progression disease, or relieving a symptom of that disease.

“Diagnose” as used herein refers to any type of administration of a diagnostic agent (such as an imaging agent or molecule) to locate, identify, or quantify a particular disease or condition, such as a tumor. The term also includes conducting a plurality of diagnoses to measure the progression of regression of the disease or condition.

“Pharmaceutically acceptable” as used herein means that the compound or composition is suitable for administration to a subject to achieve the treatments described herein, without unduly deleterious side effects in light of the severity of the disease and necessity of the treatment.

“Concurrently” as used herein means sufficiently close in time to produce a combined effect (that is, concurrently may be simultaneously, or it may mean two or more events occurring within a short time period before or after each other).

“Nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. The term nucleic acid is used interchangeably with gene, cDNA, and MRNA encoded by a gene.

“Heterologous nucleic acid” generally denotes a nucleic acid that has been isolated, cloned and ligated to a nucleic acid with which it is not combined in nature, and/or introduced into and/or expressed in a cell or cellular environment other than the cell or cellular environment in which said nucleic acid or protein may typically be found in nature. The term encompasses both nucleic acids originally obtained from a different organism or cell type than the cell type in which it is expressed, and also nucleic acids that are obtained from the same cell line as the cell line in which it is expressed.

“Nucleic acid encoding” refers to a nucleic acid which contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences which may be introduced to conform with codon preference in a specific host cell.

“Recombinant” when used with reference to a nucleic acid generally denotes that the composition or primary sequence of said nucleic acid or protein has been altered from the naturally occurring sequence using experimental manipulations well known to those skilled in the art. It may also denote that a nucleic acid or protein has been isolated and cloned into a vector, or a nucleic acid that has been introduced into or expressed in a cell or cellular environment other than the cell or cellular environment in which said nucleic acid or protein may be found in nature.

“Recombinant” when used with reference to a cell indicates that the cell replicates or expresses a nucleic acid, or produces a peptide or protein encoded by a nucleic acid, whose origin is exogenous to the cell. Recombinant cells can express nucleic acids that are not found within the native (nonrecombinant) form of the cell. Recombinant cells can also express nucleic acids found in the native form of the cell wherein the nucleic acids are re-introduced into the cell by artificial means. Such a cell is “transformed” by an exogenous nucleic acid when such exogenous nucleic acid has been introduced inside the cell membrane. Exogenous DNA may or may not be integrated (covalently linked) into chromosomal DNA making up the genome of the cell. The exogenous DNA may be maintained on an episomal element, such as a plasmid. In eucaryotic cells, a stably transformed cell is generally one in which the exogenous DNA has become integrated into the chromosome so that it is inherited by daughter cells through chromosome replication, or one which includes stably maintained extrachromosomal plasmids. This stability is demonstrated by the ability of the eucaryotic cell to establish cell lines or clones comprised of a population of daughter cells containing the exogenous DNA.

“Cell-Based Carrier” refers to cells (such as stem cells and T cells) which may be used as carriers for therapeutic molecules or other active molecules (such as a blood-brain barrier opening molecule or a tumor endothelial opening molecule). Those of skill in the drug delivery are will readily understand how to make and use such cell-based carriers.

### Heat Inducible Promoters

Any suitable heat inducible promoter may be used to carry out the present invention, examples of which include but are not limited to HSP70 promoters, HSP90 promoters, HSP60 promoters, HSP27 promoters, HSP25 promoters, ubiquitin promoters, growth arrest or DNA Damage gene promoters, etc. See, e.g., U.S. Pat. Nos. 7,186,698; 7,183,262; and 7,285,542; See also I. Bouhon et al., Cytotechnology 33: 131-137 (2000) (gad 153 promoter).

### Pro-Migratory Cytokines

A variety of pro-migratory cytokines (which may also be referred to as “stem cell-attracting chemokines,” and the nucleic acids encoding them, are known and can be used to carry out the present invention. Examples include, but are not limited to, TNF-alpha, stromal cell-derived factor 1 alpha (SDF-1 alpha), tumor-associated growth factors, transforming growth factor alpha, fibroblast growth factor, endothelial cell-derived chemoattractants, vascular endothelial growth factor (VEGF), stem cell factor (SCF), granulocyte colony-stimulating factor (G-CSF), and integrins. See, e.g., U.S. Pat. No. 8,569,471 (all of which may be mammalian, such as human).

The above list of cytokines usable in the present method is not intended to be limiting and any known cytokine or chemokine may be used in the present method as applicable to the disease or condition to be treated.

### Blood-Brain Barrier Opening and Tumor Endothelial Opening Agents

A variety of agents are known to open the blood-brain barrier in a manner beneficial to enhancing the delivery of therapeutic or diagnostic agents administered into the blood to brain tissue. Examples of such agents include, but are not limited to opening protein or peptide selected from the group consisting of bradykinin, thrombin, endothelin-1, substance P, platelet activating factor, cytokines (e.g., IL-1alpha, IL-1beta, IL-2, IL-6, TNFalpha), macrophage inflammatory proteins (e.g., MIP-1, MIP-2), and complement-derived polypeptide C3a-desArg. Similarly, a variety of agents are known to enhance the permeability of the tumor vasculature in a manner beneficial to promoting the delivery of therapeutic or diagnostic agents to the tumor.

### Therapeutic Agents

A variety of different therapeutic agents (generally protein or peptide therapeutic agents) and the nucleic acids encoding them, are known that can be used to carry out the present invention. In general, such agents are toxins, fragments of toxins, drug metabolizing enzymes, inducers of apoptosis, etc. Particular examples include, but are not limited to, bacterial toxins, plant toxins, fungal toxins and combinations thereof; kinases; and inducers of apoptosis such as PUMA; BAX; BAK; Bcl-XS; BAD; BIM; BIK; BID; HRK; Ad E1B; an ICE-CED3 protease; TNF-related apoptosis-inducing ligand (TRAIL); SARP-2; and apoptin (including active fragments thereof). See generally US Patent Application Publication No. 20130310446; see also U.S. Pat. Nos. 8,450,460; 7,972,812; 7,736,637; and 5,763,233.

### Diagnostic Agents

A variety of different diagnostic agents are known that may be used to carry out the diagnostic embodiments of the invention. Imaging agents or molecules are preferred, but other diagnostic agents may also be used as will be understood by one of skill in the art. Imaging agents are generally used to enhance contrast in images of the inside of the body obtained using X-rays, gamma rays, sound waves, radio waves (MRI), or radioactive particles to diagnose a disease or condition. See, for example, Caschera et al.—Contrast Agents in Diagnostic Imaging: Present and Future, Pharmacological Research, vol. 110, August 2016, pages 65-75.

### Recombinant Nucleic Acids and Vectors

Techniques for the production of recombinant nucleic acids, in which a promoter as described above is operatively associated with a nucleic acid encoding a pro-migratory cytokine, blood brain barrier opening agent, or therapeutic agent as described above, are known. Examples include but are not limited to those described in U.S. Pat. No. 7,186,698 to Moonen and U.S. Pat. No. 7,183,262 to Li et al.

Vectors into which such recombinant nucleic acids can be inserted, ligated, or otherwise associated, and useful for carrying out the invention are likewise known. Examples include but are not limited to DNA viral vectors, RNA viral vectors, plasmids, ballistic particles, etc.

### Stem Cells and Transformed Stem Cells

Stem cells may be stably or transiently transformed with a recombinant nucleic acid by any suitable means, with or without the use of a vector as described above. Suitable stem cells and methods and vectors for their transformation, propagation, formulation and administration are known. Examples include but are not limited to those set forth in U.S. Pat. Nos. 6,368,636; 6,387,367; 7,022,321; 8,034,329; 8,057,789; 8,216,566; and 8,518,390. The stem cells may be collected from any suitable tissue or biological fluid, such as placenta, amniotic fluid, blood, umbilical cord blood, etc. In general, the stem cells may be embryonic, adult, or induced pluripotent stem cells, with the specific choice of stem cell depending upon the specific condition and/or tissue for which they are intended.

### Pharmaceutical Formulations, Dosage and Administration

Stem cells for use in carrying out the present invention (including but not limited to those described above) may be formulated for administration in a pharmaceutically acceptable carrier in accordance with known techniques. See, e.g., Remington, The Science And Practice of Pharmacy (9.sup.th Ed. 1995). Formulations of the present invention suitable for parenteral administration comprise sterile aqueous and non-aqueous injection solutions of the active compound(s), which preparations are preferably isotonic with the blood of the intended recipient. These preparations may optionally contain anti-oxidants, buffers, bacteriostats and solutes which render the formulation isotonic with the blood of the intended recipient. Aqueous and non-aqueous sterile suspensions may include suspending agents and thickening agents.

Parenteral administration of the stem-cell containing pharmaceutical formulations may be through any suitable route, including but not limited to intraveneous, intrarterial, subcutaneous, intramuscular, and intraperitoneal injection. The number of stem cells delivered in any particular administration will depend upon a variety of factors, such as the type of stem cell being administered, the age, weight, and condition of the subject, the tissue and condition being treated, etc., but in general will be from one, five, or ten million cells, up to one, five, ten or fifty billion cells, or more.

### Tissues for Treatment

A broad variety of different tissues, including neoplastic and non-neoplastic, are known targets for stem cell treatment. See, e.g., V. Segers and R. Lee, Stem-cell therapy for cardiac disease Nature 451, 937-942 (2008); S. Kim and J. de Vellis, Stem cell-based cell therapy in neurological diseases: A review, J Neurosci. Res. 87, 2183 (2009); A. Caplan, Review: Mesenchymal Stem Cells: Cell-Based Reconstructive Therapy, in Orthopedics, Tissue Engineering, 11, 1198-1211 (2005). These tissues are also known targets for imaging.

Hence, as noted above, in some embodiments, the tissue for treatment or diagnosis (imaging) is a neoplastic or cancer tissue, examples of which include but are not limited to brain cancer tissue or tumors (e.g. gliomas such as glioblastoma multiforme, meningiomas, pituitary adenomas, nerve sheath tumors, etc.), breast cancer tissue or tumors, skin cancer tissue or tumors (e.g., melanoma, basal cell skin cancer, squamous cell skin cancer, etc.) prostate cancer tissue or tumors, lung cancer tissue or tumors, ovarian cancer tissue or tumors, colon and colorectal cancer tissue or tumors, pancreatic cancer tissue or tumors, and the like.

In other embodiments, the tissue for treatment or diagnosis (imaging) is non-neoplastic or non-cancerous tissue, but injured or diseased tissue suitable for stem cell treatment. Examples of such tissue include but are not limited to central nerve, peripheral nerve, retina, skeletal muscle, cardiac muscle, epidermal, liver, pancreatic, skeletal, endocrine, and exocrine tissue, (e.g., where the aforesaid tissue is afflicted with an acute injury, anoxic injury, metabolic disease, or autoimmune disease). Particular examples include, but are not limited to, treating or imaging acute or chronic brain injury, acute spinal-cord injury, heart damage, hematopoiesis, baldness, missing teeth, deafness, blindness and vision impairment, motor neuron diseases, graft vs. host disease, Crohn's disease, neural and behavioral birth defects, diabetes, etc.).

### Administration of HSP-Inducing Molecules

An HSP-inducing compound particularly useful in the present method is the chemotherapeutic molecule temozolomide, but it is expected that any drug that induces HSP expression will function similarly in the present method. Representative drugs useful in the present invention include: Doxorubicin 5-fluorouracil, adriamycin, cyclophosphamide, epirubicin, methotrexate, Alkannin, Osthole, Oxymatrine, Palmatine chloride, shikonin, and hypoxia inducing VEGF inhibitors like bevacizumab, sorafenib and sunitinib.

### Enhancing Blood-Brain Barrier Permeability

In one aspect of the disclosure, a method is provided to increase the blood-brain barrier permeability. Enhancing blood-brain barrier permeability is an ongoing goal (see, e.g., U.S. Pat. No. 8,349,822), and the materials and methods described herein may be used or adapted to methods of enhancing blood-brain barrier permeability, Such a method of increasing blood-brain barrier permeability of selected brain tissue in a subject in need thereof, generally comprising: (a) parenterally administering to the subject stem cells that migrate to the brain tissue, said stem cells containing a recombinant nucleic acid, said recombinant nucleic acid comprising a nucleic acid encoding a barrier-opening protein or peptide operably associated with a heat-inducible promoter; and then (b) selectively heating said selected brain tissue sufficient to induce the expression of said barrier-opening protein or peptide in an amount effective to increase the permeability of the blood-brain barrier in said selected brain tissue (e.g., so that concurrent or subsequent delivery of an active therapeutic or diagnostic agent to the selected tissue is enhanced, including but not limited to preconditioning or therapeutic stem cells as described herein, or other active agents such as therapeutic antibodies and chemotherapeutic agents).

For example, the stem cells (and the administration of the HSP-inducing molecule) can be administered in an amount effective to increase the cytotoxic effect of a therapeutic agent drug in said subject, said method further comprising administering the therapeutic agent to the subject Any suitable therapeutic agent for which enhanced BBB permeability would be advantageous may be used, examples of which include but are not limited to therapeutic stem cells (including but not limited to those described above), protein and peptide therapeutic or diagnostic agents (e.g., diagnostic and therapeutic monoclonal antibodies (including active binding fragments thereof)), or chemotherapeutic drugs. Specific examples include but are not limited to temozolomide (“Tmz”), VP-16, paclitaxel, carboplatin, tumor necrosis factor-related apoptosis-inducing ligand (“TRAIL”), troglitazone (“TGZ”), pioglitazone (“PGZ”), rosiglitazone (“RGZ”), and ciglitazone (“CGZ”), procarbazine, vincristine, BCNU, CCNU, thalidomide, irinotecan, isotretinoin, imatinib, etoposide, cisplatin, daunorubicin, doxorubicin, methotrexate, mercaptopurine, fluorouracil, hydroxyurea, vinblastine, and combinations thereof. Composition, dosage and administration of the stem cells, and heating, may be as described above, and composition, dosage and administration of the other therapeutic or diagnostic active agent may be carried out in accordance with known techniques for specific agents, or variations thereof that will be apparent to those skilled in the art. See, e.g., U.S. Pat. No. 8,450,460; see also U.S. Pat. Nos. 8,628,778; 8,580,258; 8,449,882; 8,445,216; 8,409,573; 5,624,659; and 5,558,852.

### Enhancing Tumor Permeability

In one aspect of the disclosure, a method is provided to increase the permeability of tumors to enhance the effectiveness of systemic anti-cancer therapies and imaging agents. Such a method of increasing tumor permeability permeability in a subject in need thereof, generally comprising: (a) parenterally administering to the subject stem cells, T cells or a nucleic acid that migrate to the tumor, said stem cells, T cells, or nucleic acid containing a recombinant nucleic acid, said recombinant nucleic acid comprising a nucleic acid encoding a tumor endothelial-opening molecule operably associated with a heat-inducible promoter; and then (b) administering an HSP-inducing or stress-inducing molecule sufficient to induce the expression of said tumor endothelial-opening molecule in an amount effective to increase the permeability of the tumor (e.g., so that concurrent or subsequent delivery of an active therapeutic or diagnostic agent to the tumor is enhanced, including but not limited to preconditioning or therapeutic stem cells as described herein, or other active agents such as therapeutic antibodies and chemotherapeutic agents).

For example, the stem cells, T cells or nucleic acid (and the administration of the HSP-inducing molecule) can be administered in an amount effective to increase the cytotoxic effect of a therapeutic agent drug in said subject, said method further comprising administering the therapeutic agent to the subject. Any suitable therapeutic agent for which enhanced tumor permeability would be advantageous may be used, examples of which include but are not limited to therapeutic stem cells, T cells, and nucleic acid (including but not limited to those described above), protein and peptide therapeutic or diagnostic agents (e.g., diagnostic and therapeutic monoclonal antibodies (including active binding fragments thereof)), or chemotherapeutic drugs. Specific examples include but are not limited to temozolomide (“Tmz”), VP-16, paclitaxel, carboplatin, tumor necrosis factor-related apoptosis-inducing ligand (“TRAIL”), troglitazone (“TGZ”), pioglitazone (“PGZ”), rosiglitazone (“RGZ”), and ciglitazone (“CGZ”), procarbazine, vincristine, BCNU, CCNU, thalidomide, irinotecan, isotretinoin, imatinib, etoposide, cisplatin, daunorubicin, doxorubicin, methotrexate, mercaptopurine, fluorouracil, hydroxyurea, vinblastine, and combinations thereof. Composition, dosage and administration of the stem cells, and heating, may be as described above, and composition, dosage and administration of the other therapeutic or diagnostic active agent may be carried out in accordance with known techniques for specific agents, or variations thereof that will be apparent to those skilled in the art. See, e.g., U.S. Pat. No. 8,450,460; see also U.S. Pat. Nos. 8,628,778; 8,580,258; 8,449,882; 8,445,216; 8,409,573; 5,624,659; and 5,558,852.

All documents referred to herein are incorporated herein by reference in their entirety as if fully set forth herein.

The present invention is explained in greater detail in the following non-limiting Example.

### Example

Human mesenchymal stem (hMSC) cells were infected with a previously described plasmid that expresses either Luciferase/GFP or Luciferase/TNFalpha under the HSP70 promoter. hMSCs (1×104 cells/well) were put on a 96-well plate for 24 hours before treating with 0.1 to 100 mcg/ml of temozolomide. Fluorescent microscopy was used to image GFP prior to temozolomide treatment and again after approximately 2, 5 and 24 h. Luciferase activity and expression of TNFalpha was checked after 24 h post temozolomide treatment using a Tecan SPARK luminescent reader and TNFalpha ELISA kit, respectively. Temozolomide induction of GFP was observed after about 24 hours, which was confirmed by Luciferase and TNFalpha expression, indicating temozolomide activation of the HSP70 promotor. To confirm this finding in vivo, we implanted 1×10⁶ of these infected stem cells subcutaneously in immunocompromised mice and treated ip with 1.25 mg/mouse of temozolomide. We checked for activation of luciferase 24 hours after treatment and observed luminescent signal in vivo. This activation was confirmed postmortem via imaging of activated GFP and Luciferase. We confirmed temozolomide induction as well in an orthotopic mouse model where hMSCs expressing either HSP70 TNFalpha or HSP70 GFP (control) were mixed with U251 human glioblastoma cells and implanted intracranially as we previously published and injected 72 hours later ip with 0.25 mg of temozolomide. We confirmed temozolomide activation of TNFalpha by examining for blood brain barrier permeability after perfusing animals with fluorescently labeled albumin. We found that only the temozolomide induced animals injected with HSP70 TNF alpha cells, but not induced control animals injected with HSP70 GFP cells, demonstrated selective blood brain barrier permeability in the region of stem cell implantation.

To further test the method in vivo, the infected stem cells described above were intercranially stereotatically implanted along with patient-derived (PDX) GBM cells. Five days post implantation, the mice were treated with a single therapeutic dose of temozolomide (200 mg/kg) and sacrificed 24 hours after treatment. Examining sectioned brains under a fluorescent microscope revealed significant GFP induction in the temozolomide treated mice compared to untreated controls.

To demonstrate a biologic effect, engineered stem cells that express HSP70 promotor inducible TNF alpha. We then systemically administered 200 mg/kg of temozolomide and observed blood brain darrier permeability via optical/CT imaging, which demonstrated brain deposition of systemically administered cy7-albumin, in contrast to the untreated control.

The foregoing is illustrative of the present invention, and is not to be construed as limiting thereof. The invention is defined by the following claims, with equivalents of the claims to be included therein.

1. A method of preparing a tissue for therapeutic treatment or diagnostic imaging in a subject in need thereof, comprising: (a) parenterally administering to the subject either i) a cell-based carrier that migrates to said tissue, said cell-based carrier containing a first recombinant nucleic acid or ii) a second recombinant nucleic acid that migrates to said tissue, said first and second recombinant nucleic acids comprising an encoding nucleic acid encoding a chemokine, cytokine, therapeutic molecule, or imageable molecule operably associated with a heat-inducible promoter; and (b) administering to said subject an amount of an HSP-inducing molecule sufficient to induce the expression of said chemokine, cytokine, therapeutic molecule, or imageable molecule in an amount effective to enhance the permeability of the blood-brain barrier or tumor vasculature, enhance the migration of therapeutic stem cells subsequently administered parenterally to said subject, elicit a therapeutic effect of said therapeutic molecule, or elicit an imaging effect of the imageable molecule.

2. The method of claim 1, wherein said tissue is selected from the group consisting of brain, breast, skin, prostate, lung, retina, muscle, liver, pancreatic, skeletal, and cartilage tissue.

3. The method of claim 1, wherein said tissue is a neoplastic tissue.

4. The method of claim 3, wherein said neoplastic tissue is selected from the group consisting of s brain tumor, breast cancer, skin cancer, prostate cancer and lung cancer tissue.

5. The method of claim 1, wherein said cell-based carrier is selected from the group consisting of embryonic stem cells, adult stem cells, and induced pluripotent stem cells.

6. The method of claim 1, wherein said heat inducible promoter is selected from the group consisting of an HSP70 promoter, an HSP90 promoter, an HSP60 promoter, an HSP27 promoter, an HSP25 promoter, a ubiquitin promoter, a growth arrest gene promoter, and a DNA Damage gene promoter.

7. The method of claim 1, wherein said encoding nucleic acid encodes a chemokine selected from the group consisting of TNF-alpha, stromal cell-derived factor 1alpha, tumor-associated growth factors, transforming growth factor alpha, fibroblast growth factor, endothelial cell-derived chemoattractants, vascular endothelial growth factor (VEGF), and stem cell factor (SCF).

8. The method of claim 1, wherein said parenterally administering step is a systemic administering step.

9. The method of claim 1 wherein the HSP-inducing molecule is selected from temozolomide, Doxorubicin 5-fluorouracil, adriamycin, cyclophosphamide, epirubicin, methotrexate, Alkannin, Osthole, Oxymatrine, Palmatine chloride, shikonin, bevacizumab, sorafenib and sunitinib,

10. A method of treating a tissue in a subject in need thereof, comprising: (a) parenterally administering to the subject either i) a cell-based carrier that migrates to said tissue, said cell-based carrier containing a first recombinant nucleic acid or ii) a second recombinant nucleic acid that migrates to said tissue, said first and second recombinant nucleic acids comprising an encoding nucleic acid encoding a chemokine, cytokine, or therapeutic molecule operably associated with a heat-inducible promoter; and (b) administering to said subject an amount of an HSP-inducing molecule sufficient to induce the expression of said therapeutic agent therein in a treatment-effective amount.

11. The method of claim 10, wherein said therapeutic agent is selected from the group consisting of a protein, a toxin, a fragment of a toxin, a drug-metabolizing enzyme, and an inducer of apoptosis or senescence.

12. The method of claim 10, wherein the therapeutic agent is (a) a toxin is selected from the group consisting of a bacterial toxin, a plant toxin, a fungal toxin and a combination thereof; (b) a drug-metabolizing enzyme comprising kinase; or (c) an inducer of apoptosis selected from the group consisting of PUMA; BAX; BAK; Bcl-XS; BAD; BIM; BIK; BID; HRK; Ad E1B; an ICE-CED3 protease; TRAIL; SARP-2; and apoptin.

13. The method of claim 10, wherein said tissue is brain, breast, skin, prostate, lung, retina, muscle, liver, pancreatic, skeletal, or cartilage tissue.

14. The method of claim 10, wherein said tissue is a neoplastic tissue.

15. The method of claim 14, wherein said neoplastic tissue is brain tumor, breast cancer, skin cancer, prostate cancer or lung cancer tissue.

16. The method of claim 10, wherein said therapeutic cell-based carrier is selected from embryonic stem cells, adult stem cells, and induced pluripotent stem cells.

17. The method of claim 10, wherein said heat inducible promoter is selected from the group consisting of an HSP70 promoter, an HSP90 promoter, an HSP60 promoter, an HSP27 promoter, an HSP25 promoter, a ubiquitin promoter, a growth arrest gene promoter, and a DNA Damage gene promoter.

18. The method of claim 10, wherein said parenterally administering step is a systemic administering step.

19. The method of claim 10 wherein the HSP-inducing molecule is selected from temozolomide, Doxorubicin 5-fluorouracil, adriamycin, cyclophosphamide, epirubicin, methotrexate, Alkannin, Osthole, Oxymatrine, Palmatine chloride, shikonin, bevacizumab, sorafenib and sunitinib.

20. A method of preparing for treatment and treating a tissue in a subject in need thereof, comprising: (a) parenterally administering to the subject preconditioning stem cells that migrate to said tissue, said stem cells containing a first recombinant nucleic acid, said first recombinant nucleic acid comprising a first encoding nucleic acid encoding a stem-cell attracting chemokine operably associated with a first heat-inducible promoter; (b) administering to said subject an amount of a first HSP-inducing molecule sufficient to induce the expression of said stem-cell attracting chemokine therein in an amount effective to enhance the migration of therapeutic stem cells subsequently parenterally administered to said subject; (c) parenterally administering to said subject therapeutic stem cells that migrate to said tissue, said stem cells optionally containing a second recombinant nucleic acid, said second recombinant nucleic acid comprising a second encoding nucleic acid encoding a therapeutic agent operably associated with a second heat-inducible promoter; and then optionally: (d) administering to said subject an amount of a second HSP-inducing molecule sufficient to induce the expression of said therapeutic agent from said second recombinant nucleic acid in a treatment-effective amount.

21. The method of claim 20, wherein said stem-cell attracting chemokine is selected from the group consisting of TNF-alpha, stromal cell-derived factor 1 alpha, tumor-associated growth factors, transforming growth factor alpha, fibroblast growth factor, endothelial cell-derived chemoattractants, vascular endothelial growth factor (VEGF), and stem cell factor (SCF).

22. The method of claim 20, wherein said therapeutic agent is selected from the group consisting of a protein, a toxin, a fragment of a toxin, a drug-metabolizing enzyme, and an inducer of apoptosis or senescence.

23. The method of claim 20, wherein the therapeutic agent is (a) a toxin is selected from the group consisting of a bacterial toxin, a plant toxin, a fungal toxin and a combination thereof; (b) a drug-metabolizing enzyme comprising kinase; or (c) an inducer of apoptosis selected from the group consisting of PUMA; BAX; BAK; Bcl-XS; BAD; BIM; BIK; BID; HRK; Ad E1B; an ICE-CED3 protease; TRAIL; SARP-2; and apoptin.

24. The method of claim 20, wherein said tissue is brain, breast, skin, prostate, lung, retina, muscle, liver, pancreatic, skeletal, or cartilage tissue.

25. The method of claim 20, wherein said tissue is a neoplastic tissue.

26. The method of claim 25, wherein said neoplastic tissue is brain tumor, breast cancer, skin cancer, prostate cancer or lung cancer tissue.

27. The method of claim 20, wherein said preconditioned stem cells are selected from embryonic stem cells, adult stem cells, and induced pluripotent stem cells.

28. The method of claim 20, wherein either or both said first heat inducible promoter and said second heat inducible promoter is selected from the group consisting of an HSP70 promoter, an HSP90 promoter, an HSP60 promoter, an HSP27 promoter, an HSP25 promoter, a ubiquitin promoter, a growth arrest gene promoter, and a DNA Damage gene promoter.

29. The method of claim 20, wherein either or both said parenterally administering steps is a systemic administering step.

30. A method of increasing blood-brain barrier permeability of selected brain tissue in a subject in need thereof, comprising: (a) parenterally administering to the subject stem cells that migrate to the selected brain tissue, said stem cells containing a recombinant nucleic acid, said recombinant nucleic acid comprising a nucleic acid encoding a blood brain barrier-opening protein or peptide operably associated with a heat-inducible promoter; and (b) administering to said subject an HSP-inducing molecule sufficient to induce the expression of said blood brain barrier-opening protein or peptide in an amount effective to increase the permeability of the blood-brain barrier in said selected brain tissue.

31. The method of claim 30, wherein said selected brain tissue is neoplastic tissue.

32. The method of claim 30, wherein said blood-brain barrier opening protein or peptide is selected from the group consisting of bradykinin, thrombin, endothelin-1, substance P, platelet activating factor, cytokines, macrophage inflammatory proteins, and complement-derived polypeptide C3a-desArg.

33. The method of claim 30, wherein said stem cells are selected from the group consisting of embryonic stem cells, adult stem cells, and induced pluripotent stem cells.

34. The method of claim 30, wherein said heat inducible promoter is selected from the group consisting of an HSP70 promoter, an HSP90 promoter, an HSP60 promoter, an HSP27 promoter, an HSP25 promoter, a ubiquitin promoter, a growth arrest gene promoter, and a DNA Damage gene promoter.

35. The method of claim 30, wherein said stem cells are administered in an amount effective to increase the cytotoxic effect of a therapeutic agent in said subject, said method further comprising administering the therapeutic agent to the subject.

36. The method of claim 35, wherein said therapeutic agent is selected from the group consisting of temozolomide (“Tmz”), VP-16, paclitaxel, carboplatin, tumor necrosis factor-related apoptosis-inducing ligand (“TRAIL”), troglitazone (“TGZ”), pioglitazone (“PGZ”), rosiglitazone (“RGZ”), and ciglitazone (“CGZ”), procarbazine, vincristine, BCNU, CCNU, thalidomide, irinotecan, isotretinoin, imatinib, etoposide, cisplatin, daunorubicin, doxorubicin, methotrexate, mercaptopurine, fluorouracil, hydroxyurea, vinblastine, and combinations thereof.
